(19) United States
(12) Patent Application Publication
(10) Pub. No.: US 2020/0185101 A1
(43) Pub. Date: Jun. 11, 2020

(54) DECISION SUPPORT DEVICE

(71) Applicant: Tufts Medical Center, Inc., Boston, MA (US)

(72) Inventors: Harry Selker, Wellesley, MA (US); Manlik Kwong, Corvallis, OR (US)

(21) Appl. No.: 16/598,508

(22) Filed: Oct. 10, 2019

Related U.S. Application Data
(60) Provisional application No. 62/778,084, filed on Dec. 11, 2018.

(51) Int. Cl. G16H 50/20; G16H 10/60; G16H 50/70; G16H 50/30; G16H 10/20; G06Q 50/22; G16H 50/50

(52) U.S. Cl. CPC G16H 50/20; G16H 10/60; G16H 50/70; G16H 50/50; G16H 10/20; G06Q 50/22; G16H 50/30

(57) ABSTRACT

A decision support device is used for both computation of a predictive instrument using at least some of the measurements to produce an output for presentation to a clinician, and computation of an enrollment recommendation for enrollment of the subject into at least one clinical trial for presentation to the clinician.

### CROSS-REFERENCE TO RELATED APPLICATIONS

This application claims the benefit of U.S. Provisional Application No. 62/778,084, filed Dec. 11, 2018, which is incorporated herein by reference.

This application is related to, but does not claim the benefit of, U.S. patent application Ser. No. 15/404,646, filed Jan. 12, 2017, and published as US2017/0196505, titled “PREDICTIVE INSTRUMENT TO IDENTIFY PATIENTS FOR USE OF PHARMACOLOGICAL CARDIAC METABOLIC SUPPORT,” which is incorporated herein by reference.

### STATEMENT AS TO FEDERALLY SPONSORED RESEARCH

This invention was made with government support under grant numbers TR001609, TR001064, and TR002544 awarded by the National Institutes of Health. The government has certain rights in the invention.

### BACKGROUND

This invention relates to a decision support device, and more particularly to a device and associated system for use in combination of medical diagnosis and/or treatment as well as clinical trial cohort discovery and/or clinical trial enrollment and/or trial quality control monitoring pertaining to clinical trial enrollment.

Medical decision support based on objective processing or clinical measurements has been established as a useful means of aiding diagnosis and treatment of patients. For example, mathematically and/or computationally defined transformations of clinical data to yield an output, such as a binary classification or a numerical score (e.g., a value on a continuous range, say from 0.0 to 1.0), have been used successfully. Generally, such transformations are referred to as “predictive instruments” when the output provides some prediction of the state of a patient or the predicted utility of a particular treatment. A clinician may use such a predictive instrument in making a treatment decision, for example, as described in the co-pending application cited above.

A related problem in medicine relates to testing the effectiveness of a clinical approach by conducting a clinical trial in which the approach is evaluated. Generally, if the approach is shown to be beneficial, it may be approved by the relevant regulatory bodies and adopted in clinical practice. One such approach may relate to the use of a predictive instrument, although it should be recognized that many more types of approaches are evaluated through clinical trials.

One difficult part of conducting a clinical trial relates to identification of a suitable cohort of individuals that meet a prescribed criterion for potential application of the approach to be evaluated. In general, in order for the trial to be medically and/or statistically significant, the cohort must have a suitable size and possible composition. One way to identify a cohort for a clinical trial is to identify a larger population, such as patients that arrive at the emergency room at a particular medical facility and select those patients that meet the prescribed criterion. If this approach for identifying the cohort fails to yield the needed composition of subjects, the trial may fail resulting in significant wasted effort and expense.

Therefore, there is a need to predict the effectiveness of applying selection criterion to a particular population of potential subjects, referred to as “cohort discovery” and if the predicted effectiveness is suitable, for example, in size and composition, there is a need to execute the enrollment of suitable subjects in a manner that matched the prediction.

### SUMMARY

Randomized controlled clinical trials provide the foundational evidence for the use of medical treatments. Yet delays and failures in the execution of trials mean that patients do not get access to needed medicines. The scale of the problem is daunting. Only about 10% of clinical trials achieve their projected enrollment on time and on budget. This is terrible loss of potential treatments for patients and the public, a great waste of time and money for trialists and trial sponsors, and an ethically questionable engagement of volunteers in studies that will not yield public benefit. There are many contributors to this situation, but three are especially central. First is the inaccuracy of current projection of how many study participants can be anticipated at a specific site, cohort discovery. These projections are nearly always far too optimistic, and eligible patients seem not to materialize. Second, among those eligible, enrollment is generally incomplete and biased; typical clinical trials enroll about 5-10% of all eligible patients in their target condition. Thus, enrollment takes 10-fold longer than could and those enrolled are typically the most obvious and easily accessible candidates, leading to biased samples. Third is that the monitoring of enrollment typically does not provide feedback in a way that is immediately actionable, to ensure full inclusion. Indeed, most trials do not even know the true denominator of all eligible patients for comparison to the numerator of those actually enrolled.

To address this crucial triad in an efficient, low-cost, clinically unobtrusive way, a medical device-based clinical trial approach, as described below, addresses clinical trial cohort projection, participant enrollment, and enrollment monitoring. This approach has been demonstrated with electrocardiographs for emergency and cardiac trials.

In a general aspect, embodiments described in this document describe an approach in which a decision support device serves multiple related roles of medical diagnosis and/or treatment as well as clinical trial enrollment and optional monitoring of the enrollment and/or cohort discovery. For instance, the approach includes a triad of cohort discovery for a trial, and then later trial enrollment and trial enrollment monitoring for the trial. A number of instances of such a device may be fielded in a clinical setting and may (i) provide clinical data to a central database, (ii) implement transformations of clinical data for diagnosis and/or treatment, for example, implementing one or more predictive instruments, and (iii) evaluate enrollment criteria for determining whether a subject is suitable for participation in a particular clinical trial. In at least some examples, the configuration of the device for the latter two functions may be provided from a central system, for instance, over a network.

In use in an overall medical decision support system, the clinical data provided from multiple of the decision support devices is collected in a database. Such data may then be used in characterizing cohorts of subjects that may be yielded by applying particular cohort definitions (i.e., subject selection criteria) to potential populations. When such a suitable cohort definition and population to which it should be applied is identified, it may be loaded into the decision support devices for the purpose of enrolling subjects into a corresponding study.

Another use of the clinical data collected in the database may be in defining a predictive instrument that may be used for application to subjects matching a particular cohort definition. For example, the cohort data may be used to determine a definition of a predictive instrument, for example, to configure computation and/or mathematical parameters (e.g., regression coefficients, neural network weights, and the like) to yield diagnostic and treatment outputs of the instrument. Such definitions of predictive instruments may be loaded into the decision support device for the purpose of diagnostic and treatment support to a clinician using the decision support device.

In one aspect, in general, an apparatus comprises a remote component that includes a first interface for receiving measurements from a set of clinical instruments associated with a subject; a processor configured to process the measurements, the processor being coupled to storage that stores configuration data and/or that stores a procedural specification, wherein the configuration data and/or the procedural specification are used for computation of a predictive instrument that uses at least some of the measurements to produce an output for presentation to a clinician and for computation of an enrollment recommendation for enrollment of the subject into at least one clinical trial, the enrollment recommendation being for presentation to the clinician and a second interface for communicating with a remote system via a data communication link, the second interface being part of a communication path for receipt of the configuration data and/or a procedural specification and/or for transmission of the measurements to the remote system.

In another aspect, a method comprises, at a remote component, receiving measurements from a set of one or more clinical instruments associated with a subject, using stored configuration data and/or a stored procedural specification for computing both a predictive instrument and an enrollment recommendation, the predictive instrument being configured to produce an output for presentation to a clinician based on at least some of the measurements and the enrollment recommendation being a recommendation to enroll the subject into at least one clinical trial and for presentation to a clinician, and communicating with a central component via a data communication link along which travels the configuration data and/or the procedural specification and/or the measurements.

In another aspect, in general, a medical decision support system includes a central system in data communication with a multiple remote component.

In another aspect, in general, a medical decision support method includes receiving measurements from a set of clinical instructions associated with a subject, computing a predictive instrument using at least some of the measurements to produce an output for presentation to a clinician, computing an enrollment recommendation for enrollment of the subject into at least one clinical trial for presentation to the clinician, and communicating with a remote system via a data communication link, including receiving configuration data and/or a procedural specification for use in the computing of the predictive instrument and/or in the computing of the enrollment recommendation, and/or transmitting the measurements to the remote system.

In another aspect, in general, a method for supporting a clinical trial uses a number (a potentially large number) of devices. At each of the devices subject are enrolled into the clinical trial according to an enrollment criterion stored in the device and according to measurements from a set of clinical instruments associated with said device. Enrollment of subjects at the devices is monitored, which may include processing measurements collected from the clinical instruments associated via the devices. Stored measurements collected from the clinical instruments associated with the devices are also used in cohort discovery process associated with an enrollment criterion.

In another aspect, in general, approach to the clinical research triad of study cohort discovery, study enrollment, and monitoring, focuses on the use of medical devices and other direct clinical data. Advantages of such an approach include providing a way to “integrate research into the clinical moment.” Seamlessly integrating research into usual clinical care will dramatically improve the ease, efficiency, and success of research, and importantly, it will offer patients, clinicians, and the public greater participation in clinical and healthcare research of all types. It also will provide an important capacity for hospitals and their affiliated clinical facilities to become authentic learning health systems, and thereby to improve healthcare and health. Such a system will qualitatively and quantitatively improve research beyond that now possible in healthcare systems.

A number of alternative implementations of the decision support device may be used. In some alternatives, the device is a dedicated electronic device, used in proximity to a patient and clinician supporting the patient (e.g., an emergency medical technician (EMT), emergency room doctor, etc.). Such a device may be in data communication (e.g., wired, local area wireless, etc.) with one or more clinical instruments, such as an electrocardiogram (EKG), blood oxygen sensor, etc., and may provide an output of predictive instruments and enrollment recommendations to the clinician (e.g., via printed output, visual screen, audio, etc.). Such a device would also be in data communication with a central system (e.g., continuous data communication via a wide area wireless network, local data logging with periodic data exchange with the central system, etc.). In other alternatives, the function of the decision support device may be integrated to one or more clinical instruments, provided in a server-based implementation in a computational “cloud,” distributed in some combination of being integrated into clinical instruments, implemented in a dedicated electronic device, and/or hosted in a computational cloud.

Another advantage of use of a decision support apparatus as outlined above is consistency and predictability of application on enrollment criteria for clinical trials and/or efficacy of use of predictive instruments on appropriate subjects. Such effects may improve the quality of medical care and/or reduce the cost and time needed for performing clinical trials that may bring new diagnostic or treatment approaches into general use.

Other features and advantages of the invention are apparent from the following description, and from the claims.

### DESCRIPTION OF DRAWINGS

FIG. 1 is a block diagram of a medical decision support system.

FIG. 2A is a graph plotting the observed and predicted proportion with ACI in deciles of predicted values using original validation data.

FIG. 2B is a graph plotting the observed and predicted proportion with ACI in deciles of predicted values using original development data.

FIG. 2C is a graph plotting the observed and predicted proportion with ACI in deciles of predicted values using Electronic ECG data from ACI-TIPI trial.

### DESCRIPTION

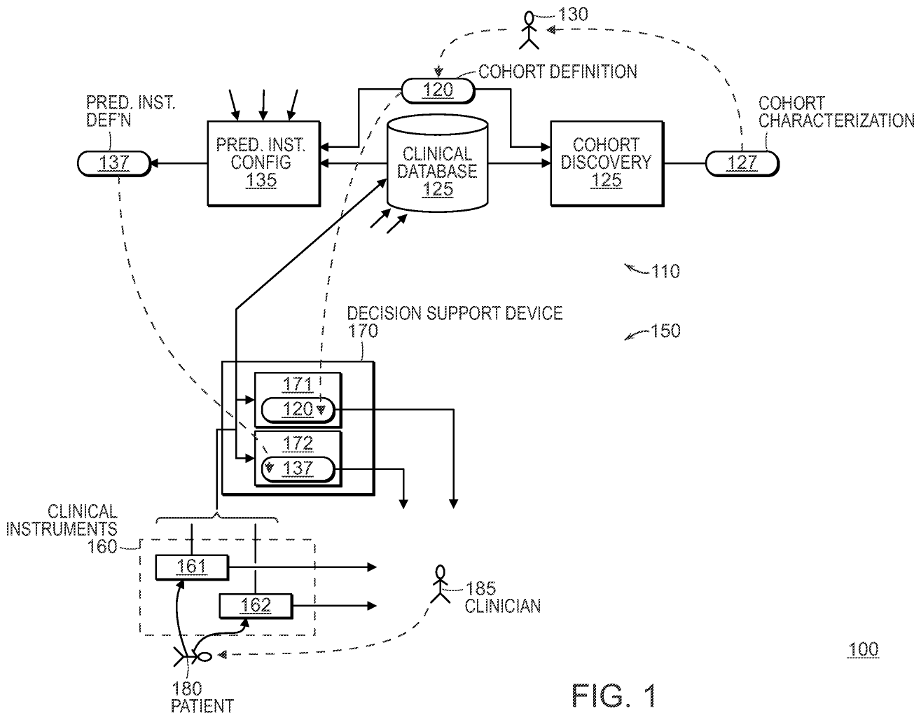

Referring to FIG. 1, a decision support system 100 includes a central component 110, and multiple remote components 150, only a representative one of which is illustrated in FIG. 1. In the representative remote component 150, a clinician 185 attends to a patient 180. A set of clinical instruments 160, including for example, an electrocardiogram (EKG) 161 and a blood oxygen sensor 162, provides measurements that are used by the clinician in making diagnosis and treatment decisions for the patient. The measurements from the set of clinical instruments 160 are provided to a decision support device 170, which provides derived information to the clinician. For example, the device 170 implements a predictive instrument 172, such as an “Acute Cardiac Ischemia Time-Insensitive Predictive Instrument” (ACI-TIPI), which is described in US2017/0196505. The predictive instrument 172 is configured with a predictive instrument definition 137, which provides configuration data and/or a procedural specification for the predictive instrument. The device 170 also includes a clinical trial enroller 171, which processes the clinical data from the set of clinical instruments 160 to output a recommendation of whether (or not) to enroll the patient 180 into a particular clinical trial. The selection criterion for the clinical trial is stored in the enroller 171 in a cohort definition 120. The clinical data is also provided from the decision support device 170 for storage in a clinical database 115.

The central component 110 of the system includes the clinical database 115, which is used for one or more functions described below. One function relates to cohort discovery. For this function, a trial administrator 130 specifies a cohort definition 120, which defines the characteristics of patients that are to be enrolled in a particular trial. A cohort discovery component 125 of the central system processes characteristics of subjects represented in the clinical database 115 according to the cohort definition 120 to characterize the cohort that meets the cohort definition. For example, the cohort characterization 127 may identify the predicted yield of suitable subjects from a particular population, such as patients that arrive at a given medical facility. To the extent that the predicted yield is sufficient, the decision support devices 170 at that given medical facility may be configured with the cohort definition 120, thereby implementing an enrollment process that should yield the predicted number of suitable subjects for the trial.

Another function of the central component 110 may be in the configuration of a predictive instrument to be applied to subjects that meet a particular cohort definition 120. For example, the clinical data 115 may be used to determine a predictive instrument definition 137 based on the clinical data 115, as well as other data such as medical outcomes resulting from diagnostic and/or treatment decisions applied to the subjects. Using these data, the predictive instrument configuration may determine configuration data, such as regression coefficients, neural network weights, decision trees, and the like, which define the input-output function of the predictive instrument. Such a predictive instrument definition may be downloaded for use in the predictive instrument 171 of the decision support device 170.

In one example of a medical device-based projection of clinical trial enrollment, electrocardiographic data was used to identify candidates for a trial in acute coronary syndromes (ACS).

To project cohorts for a trial in acute coronary syndromes, electrocardiograph-based algorithms that identify ACS or ST elevation myocardial infarction (STEMI) prompt clinicians to offer patients trial enrollment. Six hospitals' electrocardiograph systems for electrocardiograms (ECGs) meeting the planned trial's enrollment criterion were searched. ECGs with STEMI or >75% probability of ACS by the acute cardiac ischemia time-insensitive predictive instrument (ACI-TIPI).

The ACI-TIPI regression was revised to require only data directly from the electrocardiograph, the e-ACI-TIPI using the same data used for the original ACI-TIPI (development set n=3,453; test set n=2,315). In some examples, data from emergency department electrocardiographs from across the US (n=8,556) were also used. Then ACI-TIPI and e-ACI-TIPI to identify potential cohorts for the ACS trial. The cohorts identified in this manner were compared to cohorts derived from EHR data at the same hospitals.

The proposed alternative approaches identify potential study participants using data from medical devices used in real time for the clinical diagnosis that is the focus of trial enrollment. As an example, conventional computerized electrocardiographs can identify acute coronary syndromes (ACS), including acute cardiac ischemia time-insensitive predictive instrument (ACI-TIPI) predictions of ACS printed on the electrocardiogram (ECG) text header. Moreover, ST elevation myocardial infarction (STEMI) can prompt clinicians to offer patients enrollment in a trial for these conditions and has worked well for enrollment in hospital emergency department (ED) and emergency medical service (EMS) settings. Also, these electrocardiographic data can be used to monitor completeness of enrollment at trial sites. By checking the electrocardiograph management system's database, the numbers of patients actually enrolled can be compared to the denominator of all those among stored ECGs that have the qualifying features (e.g., STEMI or high ACI-TIPI probability of ACS). The ECG management database also could be used to project available patients for a clinical trial for which the electrocardiograph would be central to diagnosis, treatment, and enrollment. By searching ECG databases for patients with ECGs that qualify for enrollment, accurate projections of available cohorts is possible. In this exemplary use of the techniques, enrollment at six candidate hospitals was estimated for participation in the planned IMMEDIATE-2 Trial. Based on a previous IMMEDIATE Trial,10 IMMEDIATE-2 uses the same enrollment criteria among patients age 30 or more presenting with symptoms suggestive of ACS: having 12-lead ECGs that reflect high likelihoods of ACS (ACI-TIPI prediction of ACS of >75%) or STEMI. For device-based cohort discovery, the hospitals' ECG management systems were accessed to apply the ECG-based enrollment criteria, and for comparison, searches using the hospitals' EHR data warehouses were performed. Table 1 shows HER-based cohort discovery criteria.

### Inclusion Criteria of Group 1 and Group 2:

Criteria Group 1: ICD Code; 1 or more in record, of any of the following:

| | | |
|---|---|---|
| ICD-9 | 410* | Acute myocardial infarction, and all |
| | | downstream codes |
| ICD-9 | 411* | Other acute and subacute forms of ischemic |
| | | heart disease, and all downstream codes |
| ICD-10 | I20.0 | Unstable angina |
| ICD-10 | I21* | ST elevation (STEMI) and non-ST |
| | | elevation (NSTEMI) myocardial |
| | | infarction, and all downstream codes |

Criteria Group 2: ICD Code; 1 or more in record, of any of the following:

| | | |
|---|---|---|
| ICD-9 | 786.50 | Chest pain, unspecified |
| ICD-10 | R07.9 | Chest pain, unspecified |

### Exclusion Criteria:

Exclusion Criteria: ICD Code; 1 or more
in record, of any of the following:

| | | |
|---|---|---|
| ICD-9 | 420* | Acute pericarditis, and all downstream codes |
| ICD-10 | I30.8 | Other forms of acute pericarditis |
| ICD-10 | I30.9 | Acute pericarditis, unspecified |
| ICD-10 | I31.9 | Disease of pericardium |

For device-based cohort discovery using data collected on ED electrocardiographs, the IMMEDIATE-2 ECG inclusion criteria was applied to data acquired by the hospitals' native ED electrocardiographs (e.g., Philips PageWriter or GE Mac) stored in the hospitals' ECG data management systems (e.g., Philips TraceMaster Vue, GE MUSE, or Epiphany Cardioserver). This was done at each site to determine the number of patients meeting the criteria over a three-month period, and then the rates were annualized for enrollment projections.

To compute the ACI-TIPI probability of ACS, besides data provided by the electrocardiograph, one of the required variables is whether the patient has chest pain and whether it is the chief complaint. For enrollment in real time, this is easily obtained from the patient, as done in the original IMMEDIATE Trial. However, among the hospitals participating in this cohort projection, not all had collected this symptom report in the ED electrocardiographs when doing the first (or any) ECG. Thus, sources other than ED electrocardiographs were required for this variable. For this, the ED patient logs or hospitals' ED and/or EHR systems were used. However, from these sources, the ACI-TIPI chest pain variable levels (primary, secondary, or none) were difficult to reliably ascertain.

A new version of ACI-TIPI, “e-ACI-TIPI,” only uses electrocardiographic data or data reliably acquired in obtaining an ECG (age and gender). After deleting the variables for chest pain, the logistic regression coefficients of the original ACI-TIPI were computed using only age, gender, and ECG waveform measurements as the only variables. To allow direct comparisons of the original and modified models, the e-ACI-TIPI coefficients were generated on the same database on which ACI-TIPI had been developed The e-ACI-TIPI coefficients were then tested on the same data set on which the original ACI-TIPI was tested, using receiver-operating characteristic (ROC) curve area and calibration as metrics of performance. The full ACI-TIPI and e-ACI-TIPI were also compared using a database collected directly from ED electrocardiographs in a national trial of the use of ACI-TIPI. Additionally, the two models were compared as to the patients they identified when applying the IMMEDIATE-2 Trial inclusion criterion of >75% probability of ACS.

As a reference for comparison of the electrocardiograph-based approach to EHR-based cohort discovery, the likely IMMEDIATE-2 cohort was projected using hospitals' EHR data warehouses. Search criteria were based on codes derived through an Extraction/Transform/Load (ETL) process to provide demographics and ICD-9/ICD-10 codes that matched the target diagnoses, ACS, AMI, and STEMI (Table 1). The results were reviewed for fidelity to the intended diagnostic categories, but refined use of EHR data beyond these diagnostic codes, age, gender, and admission via the ED was beyond this project's scope.

For the EHR-based approach, the criteria in Table 1 were transformed into an SQL query for searching EHR data warehouses (or operational EHR systems), the results of which were exported into a Microsoft Excel spreadsheet for counting patients meeting target inclusion criteria and age. These data were from the same three-month period as used for electrocardiograph-based cohort discovery and were multiplied by four to generate annualized rates. At Hospital 1, as a check of the match of identification by EHRs and ED electrocardiographs, we checked a sample of EHR-identified patients to determine if they would qualify for enrollment using IMMEDIATE-2 ECG criteria.

Table 2 provides institutional characteristics including the participating hospitals' annual numbers of ED visits and numbers of patients on whom ECGs were performed in the ED.

| | | Patients over | | Mean Age | % Male |
|---|---|---|---|---|---|
| | ED Visits | 30 having a | | [of ECG | [of ECG |
| Site | per year | 12-lead ECG | Setting | sample] | sample] |
| H1 | 40,000 | 8,212 | Large urban | 60 | 52% |
| | | | setting | | |
| H2 | 110,000 | 22,124 | Small city | 55 | 46% |
| H3 | 69,565 | 22,328 | Moderate | 65 | 51% |
| | | | sized city | | |
| H4 | 36,000 | 13,152 | Large urban | 57 | 49% |
| | | | setting | | |
| H5 | 72,000 | 12,336 | Very large city | 64 | 50% |
| H6 | 70,340 | 17,544 | Very large city | 63 | 57% |

The logistic regression coefficients for the original ACI-TIPI and e-ACI-TIPI are in Table 3 below. These logistic regression coefficients were estimated from ACI-TIPI development database of n=3,453 subjects, of whom 1,251 had ACS.

| | | Model Coefficients | |
|---|---|---|---|
| | | Original ACI- | e-ACI- |
| Variable | Variable Description | TIPI | TIPI |
| CONSTANT | Intercept | −3.9327 | −2.2446 |
| SX1CPAIN | Chief Complaint of Chest Pain | 0.8817 | — |
| CPAIN | Chest Pain | 1.2308 | — |
| MALESEX | Male Gender | 0.7121 | 0.7253 |
| AGE40 | Patient age 40 years of less | −1.4408 | −1.3281 |
| AGE50 | Patient age over 50 years | 0.6673 | 0.4514 |
| SEXAGE50 | Male patient over 50 years old | −0.4265 | −0.4589 |
| QWAVE | ECG Q waves present | 0.6158 | 0.5261 |
| STEL | ECG S-T segment elevation | 1.3141 | 1.2572 |
| | (2 if >2 mm, 1 if | | |
| | 1-2 mm, 0 otherwise) | | |
| STDEP | ECG S-T segment depression | 0.9933 | 0.9612 |
| | (2 if >2 mm, 1 if | | |
| | 1-2 mm, 0.5 if 0.5-1 mm, | | |
| | 0 otherwise) | | |
| TWEL | ECG T-waves elevated | 1.0952 | 1.1218 |
| | (1 if hyperacute/elevated, | | |
| | 0 otherwise) | | |
| TWINV | ECG T-wave inversion | 1.1270 | 1.0789 |
| | (2 if inverted 5 mm or more, | | |
| | 1 if inverted 1-5 mm, | | |
| | 0.5 if flat, 0 otherwise) | | |
| TWISTDEP | 1 if both STDEP and TWINV | −0.3140 | −0.2964 |
| | not 0, 0 otherwise | | |

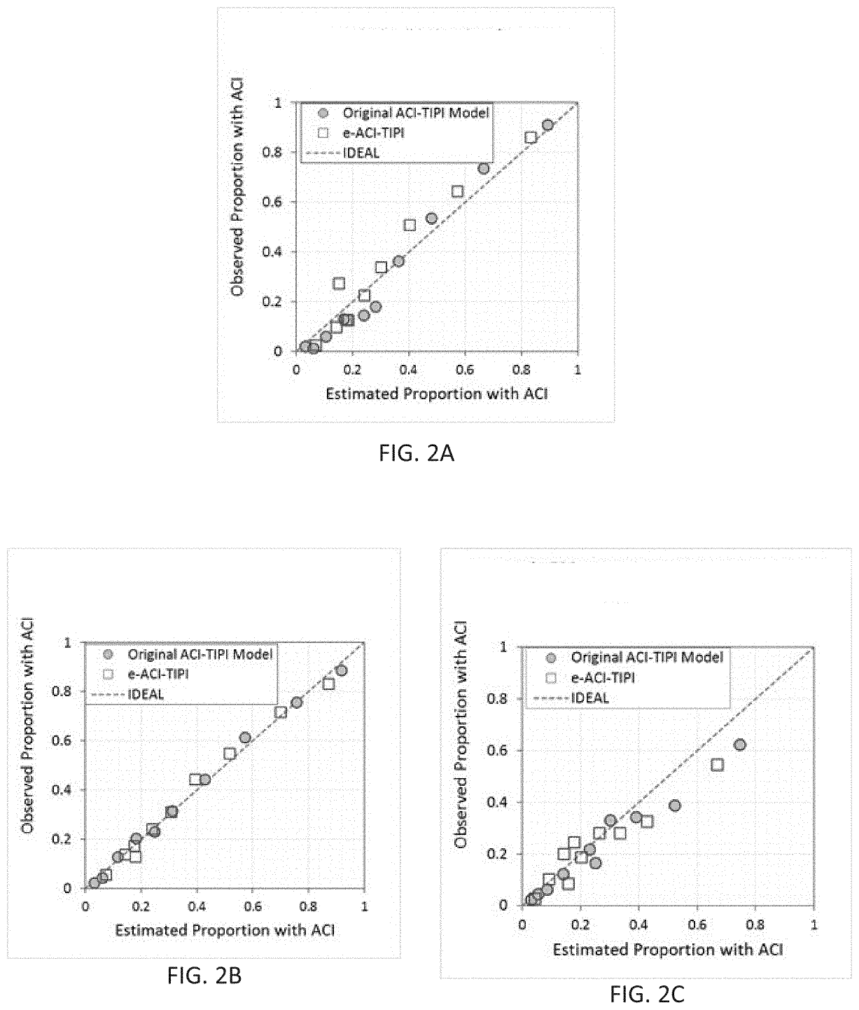

The ROC areas for ACI-TIPI and e-ACI-TIPI, on development and test data sets, are shown in Table 4 below; their calibration graphs are shown in FIG. 2. The ROC areas were slightly less in the e-ACI-TIPI compared to the ACI-TIPI, both in the development and test data sets, but being at or above 0.8 in all cases reflected excellent diagnostic performance. When applied to data collected from electrocardiographs in EDs nationally, the ROC for e-ACI-TIPI was lower, at 0.69, but still reflecting very good performance.

| | | | Original | |
|---|---|---|---|---|
| | | | ACI-TIPI | e-ACI-TIPI |
| | Sample | Prevalence | model | model |
| COHORT | Size | of ACS | ROC Areas | |
| Original | 3,453 | 36.2% | 0.85 | 0.80 |
| development data | | | | |
| Original validation | 2,315 | 30.7% | 0.89 | 0.84 |
| data set | | | | |
| Electronic ECG data | 8,556 | 23.5% | 0.78 | 0.69 |
| from ACI-TIPI Trial | | | | |

The comparisons of results of applying the IMMEDIATE inclusion criterion of >75% using the original ACI-TIPI and e-ACI-TIPI are shown in Table 5 below.

Comparison of Proportions of Cases Identified as Having ≥75% Probability of ACS

| | % with ≥75% for ACS | | | | |
|---|---|---|---|---|---|
| Datasets | Original TIPI | | e-ACI-TIPI | | p-value |
| Original development data | 14.9% | (515/3,453) | 12.5% | (433/3,453) | <.0001 |
| Original validation data set | 10.9% | (252/2,315) | 8.2% | (189/2,315) | <.0001 |
| Electronic ECG data from ACI- | 4.2% | (358/8,556) | 2.5% | (217/8,556) | <.0001 |
| TIPI Trial | | | | | |

Agreement Between Original TIPI and e-ACI-TIPI on
Cases Identified as Having ≥75% Probability of ACS

| Datasets | All Cases | | True Non-ACS | | True ACS | |
|---|---|---|---|---|---|---|
| Original development data | 93.8% | (3,239/3,453) | 97.7% | (2,152/2,202) | 86.9% | (1,087/1,251) |
| Original validation data set | 95.6% | (2,212/2,315) | 98.5% | (1,580/1,604) | 88.9% | (632/711) |
| Electronic ECG data from ACI- | 97.5% | (8,339/8,556) | 98.5% | (6,448/6,543) | 93.9% | (1,891/2,013) |
| TIPI Trial | | | | | | |

Although statistically significantly different, the proportions identified by each approach were very close. The e-ACI-TIPI detected about 20% fewer patients, and thus provides conservative estimates of patient numbers that the full ACI-TIPI would detect in real-time care.

Referring to Table 6, cohort discovery findings (i.e., numbers of patients identified, annualized) were collected among patients older than age 30 and admitted via EDs using EHR- and Electrocardiograph-based Projections. These cohort discovery results were based on three-month assessments and were expressed as one-year estimates to project potential accrual over one year.

TABLE 6

| | | ECG Cohort | ECG Cohort | Patients Identified |
|---|---|---|---|---|
| | EHR Cohort | Discovery Using | Discovery Using | by both EHR |
| Site | Discovery | ACI-TIPI | e-ACI-TIPI | and ECG Methods |
| H1 | 32 | 8* | 28 | 0 overlap by both EHR and |
| | | | | ACI-TIPI |
| | | | | 4 overlap by both EHR and |
| | | | | e-ACI-TIPI |
| H2 | 652 | 284 | 316 | 44 overlap by both EHR and |
| | | | | ACI-TIPI |
| | | | | 56 overlap by both EHR and |
| | | | | e-ACI-TIPI |
| H3 | 540 | 400 | 448 | NA |
| H4 | 640 | 136 | 256 | NA |
| H5 | 128 | 124 | 216 | NA |
| H6 | 1,680 | 1,980** | 660 | NA |
| H6 | 1,680 | 636*** | 660 | NA |
| H6 | 1,680 | 224* | 660 | NA |

*Using “no chest pain” for ACI-TIPI calculation
**Using “primary chest pain” status for ACI-TIPI calculation
***Using “secondary chest pain” status for ACI-TIPI calculation

The EHR-based cohort projections for patients with ACS had wide differences in total counts across different hospitals, and the estimates across hospitals made by the e-ACI-TIPI were more consistent. Patient discovery using the ECG- and EHR-based methods were compared at the two hospitals at which sufficiently detailed data were available. There was found to be little overlap between the ECG- and EHR-based cohorts. A suggestion of the cause of the discrepancy was obtained from clinical reviews done at Hospital 1, where among 16 EHR-identified patients, 14 had ECGs done in the ED, of which only one met IMMEDIATE-2 ECG enrollment criteria.

Also reflected in Table 6, the cohort estimates based on the ACI-TIPI were compared to the e-ACI-TIPI, looking for the influence of variation in the chest pain variable, which is present in ACI-TIPI but not in e-ACT-TIPI. No hospital had uniform presence of electronic data on this variable, which were derived at each site using the best available data, which had limitations. At Hospital 1, the ED Director considered the chief complaint recorded on the electrocardiograph as unreliable, and so as a default, we made the most conservative assumption that would lower the estimate of the ACS cohort, that is, that the patients did not have chest pain. Hospital 2 did have data from their ED records of chief complaints and reasons for ED visits, from which we could infer the three-level variable, chest pain as a primary complaint, a secondary complaint, or not present. For Hospital 3, the presence of chest pain, not differentiated as primary or secondary, was based on the ED chief complaint. For Hospital 4, the variable was derived from the reason the ECG was ordered, simply chest pain present or absent. Also, for Hospital 5, the chest pain variable was the reason the ECG was ordered, but from a short list of potential reasons: six different chest pain-related reasons, compared to more than 70 chest pain-related reasons at Hospital 4. At Hospital 6, there were no chest pain data, and so, in part to illustrate the range that this variable could induce, we simulated the ACI-TIPI each as if patients had a chief complaint of chest pain, a secondary complaint of chest pain, or no chest pain, shown in the last three rows of Table 6.

This uncertainty and variety in the ACI-TIPI chest pain variable were reflected in the differences between the cohorts identified by it and the e-ACI-TIPI. In the case of Hospital 2, where two independent EHR documentation fields were available and used (chief complaint and reason for visit), cohort discovery was more consistent between the ACI-TIPI (284 patients) and e-ACI-TIPI (316 patients), compared to the other hospitals at which only a single EHR field (chief complaint) was available (ACI-TIPI vs e-ACI-TIPI being, respectively, 8 vs 28 patients; 400 vs 448 patients; 136 vs 256 patients; and 124 vs 216 patients). This finding illustrates the variability in local EHR documentation and shows the e-ACI-TIPI to be a more consistent instrument for cohort discovery across varied ED environments and EHR documentation practices. At Hospital 6, where the full ACI-TIPI could not be computed because of the absence of data for the chest pain variable, the range of projections by simulation using three levels of the chest pain variable (primary, secondary, or none) were, respectively, 1980 patients, 636 patients, and 224 patients. This illustrates the impact of removing the chest pain variable from the ACI-TIPI model for cohort projection.

The above described approach can also be applied to other medical instruments and clinical trial focus in the examples below.

With reference to FIG. 1, the approach can be applied to over-use of C-sections. In such a trial, patients are first identified retrospectively through cohort discovery (125) using a clinical database (115) to characterize those patients that had a C-section with positive and negative outcomes (child and/or mother). Similar to the ACS example, the mother's demographics and fetal monitor during labor (160) data can be obtain and used to define the patient cohort definition (120). Thresholds (simple prediction analytics—172) associated with the medical instrument (fetal monitor—161) can then be determined from a study of the patient cohort and its definition in order to then be utilized in the decision support device (170) to enroll patients into a prospective trial to help reduce over use of C-sections.

In another example, the approach is applied to Patent Foramen Ovale (PFO) for Secondary Stroke Prevention. In such a trial, patients are first identified through retrospective cohort discovery (125) using a clinical database (115) to characterize those patients that were diagnosed with Pulmonary Embolism (PE), had a diagnosis of stroke, and positive finding of PFO by transcranial doppler (161) or transesophageal echocardiography (161). Predictive analytics (172) can then be derived from the characteristics of the study cohort and its medical instrumentation (echocardiograph—160) and clinical trial inclusion thresholds set (170) to identify and enroll patients into a clinical trial.

In yet another example, the approach is applied to Knee Osteoarthritis (OA). In such a trial, patients are first identified through retrospective cohort discovery (125) using a clinical database (115) to characterize patients that have complaint of knee pain, of certain demographics (e.g., age—older patient, weight), and X-ray (161) findings. X-ray findings may include derivation of knee-gap measurements as well as assessment of osteoarthritis and other findings suggestive of appropriateness of surgical or non-surgical medical intervention. Predictive analytics (172) can therefore be derived from the cohort characterization (120) to establish enrollment criteria and decision making (170) for enrollment for example of patients with and without knee surgery versus non-surgical approaches.

It should be understood that the arrangement illustrated in FIG. 1 may have a number of alternative implementations. For example, the functionality of the decision support device 170 may be integrated with one or more of the clinical instruments 160. Alternatively, the devices 170 are “add-on” devices to existing clinical instructions 160 without requiring modification of those devices. Some of the functionality of the support device 170 may be hosted in a central component. For example, the enrollment function of the device 170 may be hosted in the central component, and the cohort discovery process may be hosted on one or more or the devices 170. Yet other implementations that provide the same or similar functionality as shown in the arrangement of FIG. 1 may be used.

The decision support device 170 may be implemented in software, in hardware, or in a combination of software and hardware. In some alternatives, the decision support device is a dedicated electronic device (e.g., with application-specific circuitry and/or a programmable processor), used in proximity to a patient and clinician supporting the patient (e.g., an EMT, emergency room doctor, etc.). Such a device may be in data communication (e.g., wired, local area wireless, etc.) with one or more clinical instruments, such as an EKG, blood oxygen sensor, etc., and may provide an output of predictive instruments and enrollment recommendations to the clinician (e.g., via printed output, visual screen, audio, etc.). Such a device would also be in data communication with a central system (e.g., continuous data communication via a wide area wireless network, local data logging with periodic data exchange with the central system, etc.). In other alternatives, the function of the decision support device may be integrated to one or more clinical instruments, provided in a server-based implementation in a computational “cloud,”, distributed in some combination of being integrated into clinical instruments, implemented in a dedicated electronic device, and/or hosted in a computational cloud.

It is to be understood that the foregoing description is intended to illustrate and not to limit the scope of the invention, which is defined by the scope of the appended claims. Other embodiments are within the scope of the following claims.

What is claimed is:

1. An apparatus comprising a remote component (150), the remote component comprising:
a first interface for receiving measurements from a set of clinical instruments associated with a subject;
a processor configured to process the measurements, the processor being coupled to storage that stores configuration data and/or that stores a procedural specification, wherein the configuration data and/or the procedural specification are used
for computation of a predictive instrument that uses at least some of the measurements to produce an output for presentation to a clinician and
for computation of an enrollment recommendation for enrollment of the subject into at least one clinical trial, the enrollment recommendation being for presentation to the clinician; and
a second interface for communicating with a remote system via a data communication link, the second interface being part of a communication path for receipt of the configuration data and/or a procedural specification and/or for transmission of the measurements to the remote system.

2. The apparatus of claim 1, further comprising a central system, wherein the remote component is one of a plurality of identically-structured remote components, all of which are in data communication with the central system, wherein the remote components and the central system together defined a medical decision support system.

3. The apparatus of claim 1, wherein the set of clinical instruments comprises an EKG.

4. The apparatus of claim 1, wherein the set of clinical instruments comprises a blood-oxygen sensor.

5. The apparatus of claim 1, further comprising a decision-support device for implementing the predictive instrument, wherein the first interface provides the measurements to the decision-support device.

6. The apparatus of claim 5, wherein the decision-support device stores the measurements.

7. The apparatus of claim 5, wherein decision-support device is configured to implement an acute cardiac ischemia time-insensitive predictive instrument.

8. The apparatus of claim 5, wherein the predictive instrument comprises a predictive-instrument definition that provides at least one of configuration data and a procedural specification for the predictive instrument.

9. The apparatus of claim 5, wherein the decision-support device comprises a clinical-trial enroller in data communication with one or more of the clinical instruments, the clinical-trial enroller being configured to use data provided by the one or more clinical instruments and data in a stored cohort definition to output a recommendation, the recommendation being indicative of a suitability of the subject for enrollment in a particular clinical trial for which the cohort definition provides criteria for enrollment.

10. The apparatus of claim 5, wherein the decision-support device is integrated into one of the clinical instruments.

11. The apparatus of claim 2, wherein the central component comprises a cohort-discovery component that is configured to carry out cohort discovery using a cohort definition that defines characteristics of patients that are to be enrolled in a particular trial.

12. The apparatus of claim 10, wherein the cohort-discovery component is configured to predict a yield of suitable subjects from a first population, said suitable subjects being those subjects that possess the characteristics of patients that are to be enrolled in the particular trial.

13. The apparatus of claim 11, wherein the cohort-discovery component is further configured to determine that the predicted yield exceeds a threshold of sufficiency and, after having done so, to configure one or more decision-support devices that are constituents of one or more remote components that serve the first population, thereby implementing an enrollment process for enrolling patients in the particular trial.

14. The apparatus of claim 5, further comprising a cohort-discovery process that is implemented on the decision-support device, wherein the cohort-discovery component is configured to carry out cohort discovery using a cohort definition that defines characteristics of patients that are to be enrolled in a particular trial

15. The apparatus of claim 2, further comprising a decision-support device for implementing the predictive instrument, wherein the decision-support device is implemented on the central component.

16. The apparatus of claim 1, further comprising application-specific circuitry configured to implement a decision-support device for implementing the predictive instrument, wherein the first interface provides the measurements to the application-specific circuitry.

17. The apparatus of claim 2, wherein the central system is configured to determine the predictive instrument based on at least clinical data to determine configuration data selected from the group consisting of regression coefficients, neural-network weights, and decision trees, wherein the configuration data defines an input-output function of the predictive instrument for download to a decision-support device at the remote component.

18. A method comprising, at a remote component,
receiving measurements from a set of one or more clinical instruments associated with a subject,
using stored configuration data and/or a stored procedural specification for computing both a predictive instrument and an enrollment recommendation, the predictive instrument being configured to produce an output for presentation to a clinician based on at least some of the measurements and the enrollment recommendation being a recommendation to enroll the subject into at least one clinical trial and for presentation to a clinician, and
communicating with a central component via a data communication link along which travels the configuration data and/or the procedural specification and/or the measurements.

19. A method for carrying out medical decision support, the method comprising:
receiving measurements from a set of clinical instruments (160) associated with a subject;
computing a predictive instrument using at least some of the measurements to produce an output for presentation to a clinician;
computing an enrollment recommendation for enrollment of the subject into at least one clinical trial for presentation to the clinician; and
communicating with a remote system via a data communication link, wherein communicating with the remote system includes
receiving configuration data and/or a procedural specification for use in the computing of the predictive instrument and/or in the computing of the enrollment recommendation, and/or
transmitting the measurements to the remote system.

20. A method comprising supporting a clinical trial using a plurality of devices, wherein supporting the clinical trial comprises:
at each of the plurality of devices, enrolling a subject into the clinical trial based on enrollment-criterion data stored in the device and measurements from a set of clinical instruments associated with said device;
monitoring enrollment of subjects at the devices, wherein monitoring enrollment includes processing measurements collected from the clinical instruments associated with the devices; and
using stored measurements collected from the clinical instruments associated with the devices in a cohort discovery process associated with an enrollment criterion.
